(19) United States
(12) Patent Application Publication
(10) Pub. No.: US 2023/0073132 A1
(43) Pub. Date: Mar. 9, 2023

(54) ANTI-CD 160 BINDING MOLECULES FOR TREATING DISEASES

(71) Applicant: MEDSTAR HEALTH, INC., Columbia, MD (US)

(72) Inventors: Stephen E. Epstein, Columbia, MD (US); Dror Luger, Columbia, MD (US); Michael Lipinski, Columbia, MD (US)

(21) Appl. No.: 17/787,959

(22) PCT Filed: Dec. 30, 2020
(86) PCT No.: PCT/US2020/067448

§ 371 Date: Jun. 22, 2022

Related U.S. Application Data
(60) Provisional application No. 62/955,569, filed on Dec. 31, 2019.

(51) Int. Cl. C07K 16/28; A61P 37/06

(52) U.S. Cl. CPC C07K 16/2803; A61P 37/06; A61K 2039/505

(57) ABSTRACT

The present invention relates to CD160 binding molecules and nucleic acid sequences encoding such molecules. In particular embodiments, the present invention provides CD160 binding molecules (e.g., monoclonal antibodies or antibody fragments) with particular light chain and/or heavy chain CDRs (e.g., selected from SEQ ID NOS:2-4, 10-12, 6-8, and 14-16) and methods for using such molecules to treat an inflammatory disease, cancer, a chronic infection, and a refractory infection, or to determine whether cells with the CD160 surface receptor are present in certain tissues or cells being examined.

### CROSS-REFERENCE TO RELATED APPLICATIONS

This application claims priority to U.S. Provisional Patent Application No. 62/955,569, filed on Dec. 31, 2019, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Dec. 23, 2020, is named MSH-029959 WO ORD SEQUENCE LISTING_ST25 and is 9,138 bytes in size.

### FIELD OF THE INVENTION

The present invention relates to CD160 binding molecules and nucleic acid sequences encoding such molecules. In particular embodiments, the present invention provides CD160 binding molecules (e.g., monoclonal antibodies or antibody fragments) with particular light chain and/or heavy chain CDRs (e.g., selected from SEQ ID NOS:2-4, 10-12, 6-8, and 14-16) and methods for using such molecules to treat an inflammatory disease, cancer, a chronic infection, and a refractory infection, or to determine whether cells with the CD160 surface receptor are present in certain tissues or cells being examined

### BACKGROUND OF THE INVENTION

An innate inflammatory response is a reaction mounted by the immune system in response to an injury, infection, or threat. It is distinguished from the more precisely tailored adaptive responses of the immune system. Inflammation may work cooperatively with adaptive responses of the immune system, which develop more slowly but are more precisely targeted to a harmful agent such as a pathogen that may be causing localized injury. While associated with infection, inflammation occurs in response to many types of injury, including physical trauma, burns (e.g., from radiation, heat or corrosive materials), chemical or particulate irritants, bacterial or viral pathogens, and localized oxygen deprivation (ischemic). Inflammation is also associated with autoimmune diseases and allergic reactions. Inflammation includes the classic symptoms of redness, heat, swelling, and pain, and may be accompanied by decreased function of the inflamed organ or tissue. In many instances inflammation, an essential response to prevent damage and death from infection, can be excessive and persistent, and thereby become a cause of damage and dysfunction of a range of tissues and organs. While a number of methods for treating this excessive inflammation are known, all of them have limitations, particularly with regard to broad-based efficacy. Thus, there is a need for new methods for reducing, alleviating and/or preventing the kind of excessive, persistent inflammation associated with a variety of diseases.

### SUMMARY OF THE INVENTION

The present invention relates to CD160 binding molecules and nucleic acid sequences encoding such molecules. In particular embodiments, the present invention provides CD160 binding molecules (e.g., monoclonal antibodies or antibody fragments) with particular light chain and/or heavy chain CDRs (e.g., selected from SEQ ID NOS:2-4, 10-12, 6-8, and 14-16) and methods for using such molecules to treat an inflammatory disease, cancer, a chronic infection, and a refractory infection, or to determine whether cells with the CD160 surface receptor are present in certain tissues or cells being examined

In some embodiments, provided herein are compositions comprising a CD160 binding molecule (e.g., a purified CD160 binding molecule), wherein the CD160 binding molecule comprises: a) a light chain variable region, wherein the light chain variable region comprises; i) a CDRL1 amino acid sequence selected from the group consisting of SEQ ID NO:2, SEQ ID NO:10, SEQ ID NO:2 with one or more substitutions from Table 1A and/or with one or more conservative amino acid changes, and SEQ ID NO:10 with one or more substitutions from Table 3A and/or one or more conservative amino acid changes; ii) a CDRL2 amino acid sequence selected from the group consisting of SEQ ID NO:3, SEQ ID NO:11, SEQ ID NO:3 with one or more substitutions from Table 1B and/or with one or more conservative amino acid changes, and SEQ ID NO:11 with one or more substitutions from Table 3B and/or one or more conservative amino acid changes; and iii) a CDRL3 amino acid sequence selected from the group consisting of SEQ ID NO:4, SEQ ID NO:12, SEQ ID NO:4 with one or more substitutions from Table 1C and/or with one or more conservative amino acid changes, and SEQ ID NO:12 with one or more substitutions from Table 3C and/or one or more conservative amino acid changes; and b) a heavy chain variable region, wherein the heavy chain variable region comprises: i) a CDRH1 amino acid sequence selected from the group consisting of SEQ ID NO:6, SEQ ID NO:14, SEQ ID NO:6 with one or more substitutions from Table 2A and/or with one or more conservative amino acid changes, and SEQ ID NO:14 with one or more substitutions from Table 4A and/or with one or more conservative amino acid changes; ii) a CDRH2 amino acid sequence selected from the group consisting of SEQ ID NO:7, SEQ ID NO:15, SEQ ID NO:7 with one or more substitutions from Table 2B and/or with one or more conservative amino acid changes, and SEQ ID NO:15 with one or more substitutions from Table 4B and/or with one or more conservative amino acid changes; and iii) a CDRH3 amino acid sequence selected from the group consisting of SEQ ID NO:8, SEQ ID NO:16, SEQ ID NO:8 with one or more substitutions from Table 2C and/or with one or more conservative amino acid changes, and SEQ ID NO:16 with one or more substitutions from Table 4C and/or with one or more conservative amino acid changes.

In certain embodiments, provided herein are compositions comprising at least one of the following: a) a first nucleic acid sequence (e.g., an isolated first nucleic acid sequence) encoding a light chain variable region, wherein the light chain variable region comprises: i) a CDRL1 amino acid sequence selected from the group consisting of SEQ ID NO:2, SEQ ID NO:10, SEQ ID NO:2 with one or more substitutions from Table 1A and/or with one or more conservative amino acid changes, and SEQ ID NO:10 with one or more substitutions from Table 3A and/or one or more conservative amino acid changes; ii) a CDRL2 amino acid sequence selected from the group consisting of SEQ ID NO:3, SEQ ID NO:11, SEQ ID NO:3 with one or more substitutions from Table 1B and/or with one or more conservative amino acid changes, and SEQ ID NO:11 with one or more substitutions from Table 3B and/or one or more conservative amino acid changes; and iii) a CDRL3 amino acid sequence selected from the group consisting of SEQ ID NO:4, SEQ ID NO:12, SEQ ID NO:4 with one or more substitutions from Table 1C and/or with one or more conservative amino acid changes, and SEQ ID NO:12 with one or more substitutions from Table 3C and/or one or more conservative amino acid changes; and/or b) a second nucleic acid sequence (e.g., an isolated second nucleic acid sequence) encoding a heavy chain variable region, wherein the heavy chain variable region comprises: i) a CDRH1 amino acid sequence selected from the group consisting of SEQ ID NO:6, SEQ ID NO:14, SEQ ID NO:6 with one or more substitutions from Table 2A and/or with one or more conservative amino acid changes, and SEQ ID NO:14 with one or more substitutions from Table 4A and/or with one or more conservative amino acid changes; ii) a CDRH2 amino acid sequence selected from the group consisting of SEQ ID NO:7, SEQ ID NO:15, SEQ ID NO:7 with one or more substitutions from Table 2B and/or with one or more conservative amino acid changes, and SEQ ID NO:15 with one or more substitutions from Table 4B and/or with one or more conservative amino acid changes; and iii) a CDRH3 amino acid sequence selected from the group consisting of SEQ ID NO:8, SEQ ID NO:16, SEQ ID NO:8 with one or more substitutions from Table 2C and/or with one or more conservative amino acid changes, and SEQ ID NO:16 with one or more substitutions from Table 4C and/or with one or more conservative amino acid changes. In some embodiments, the compositions further comprise an expression vector, and wherein the first and/or second nucleic acid sequences are present in the expression vector. In other embodiments, the light and heavy chain variable regions are part of a CD160 binding molecule.

In particular embodiments, the CD160 binding molecule is an antibody, minibody, diabody, scFv, or antibody fragment capable of binding human CD160. In other embodiments, the antibody fragment is a Fab or Fv antibody fragment. In other embodiments, the antibody fragment comprises an antigen binding portion of the G6C9 or 4C10 antibody. In further embodiments, the light and/or heavy chain variable region comprises a mouse or human framework region.

In additional embodiments, the CD160 binding molecule further comprises a light chain constant region and a CH1 heavy chain constant region. In some embodiments, the CD160 binding molecule further comprises a CH2 heavy chain constant region and/or a CH3 heavy chain constant region. In further embodiments, the CD160 binding molecule is an antibody or antigen binding portion thereof that has an Fc region characterized in that it: i) has an Fc cellular binding site; and/or ii) has a Fc complement binding site; and/or 3) has a linked toxin that is internalized into a cell. In additional embodiments, the light chain constant region is human or a humanized murine, and/or wherein the CH1, CH2, and CH3 heavy chain constant regions are human or are humanized murine.

In particular embodiments, the CD160 binding molecule comprises an antibody, wherein the light chain constant region of the antibody is selected from: IgG Kappa and IgG Lambda, and wherein the heavy chain constant region of the antibody is selected from: IgG1, IgG2, IgG3, and IgG4. In certain embodiments, the CD160 binding molecule comprises an antibody, or antigen binding portion thereof, which is fucosylated or non-fucosylated. In other embodiments, the compositions further comprise a physiologically tolerable buffer.

In some embodiments, provided herein are methods of treating or preventing a disease or infection comprising: treating a subject with a CD160 binding molecule, or an expression vector encoding said CD160 binding molecule, and wherein the subject has, or is suspected to develop, a disease or infection selected from: an inflammatory disease, cancer, a chronic infection, and a refractory infection (e.g., heart valve infections, viral infection, or abscesses), or to determine whether cells with the CD160 surface receptor are present in certain tissues or cells being examined When used to treat or prevent a disease or infection, the CD160 binding molecules can be used to deplete and/or inhibit NK cells, thereby decreasing inflammation.

In certain embodiments, the CD160 binding molecules are as recited above and herein. In some embodiments, the CD160 binding molecules are selected from one of the following anti-human CD160 monoclonal antibodies: CL1-R2, (MBL Int.), LS-C179644 (LS Bio), Clone # 688327 (R&D Systems), and Clone 9i94 (MyBioSource.com).

In certain embodiments, the inflammatory disease is a cardiovascular disease or autoimmune disease. In further embodiments, the cardiovascular disease is selected from: heart failure, atherosclerosis, progression of atherosclerotic disease, cardiogenic shock, and inflammation post treatment with an immune checkpoint inhibitor. In additional embodiments, the disease is a cardiovascular disease (e.g., Coronary Heart Disease, Cardiomyopathy, Myocardial Infarction, Congestive Heart Failure. In certain embodiments, the autoimmune disease is selected from, but not limited to, the group consisting of: rheumatoid arthritis, inflammatory bowel disease, septic shock, and psoriasis.

In further embodiments, the CD160 binding molecule is an antibody or antigen binding portion thereof. In some embodiments, the antibody or antigen binding portion thereof is a human antibody or antigen binding portion thereof. In certain embodiments, the antibody or antigen binding portion thereof is a humanized antibody or antigen binding portion thereof. In additional embodiments, a sample from the subject is assayed to determine levels of NK cell activation and/or inflammation before and/or after the treating.

In some embodiments, provided herein are methods of detecting CD160 in a sample comprising: a) contacting a sample with the CD160 binding molecule as described herein, wherein the sample is from a subject that has, or is suspected to develop, a disease or infection selected from: an inflammatory disease, cancer, a chronic infection, and a refractory infection, wherein the sample is suspected of containing CD160, and wherein the CD160 binding molecule forms a complex with the CD160 if present in the sample; and b) detecting the presence or absence of the complex in the sample. In other embodiments, the CD160 binding molecule comprises a detectable label. In additional embodiments, the methods further comprise contacting the sample with a conjugate molecule capable of binding to the CD160 binding molecule, wherein the conjugate molecule comprises a detectable label.

### BRIEF DESCRIPTION OF THE FIGURES

The present invention may be more readily understood by reference to the following figures, wherein:

FIG. 1 shows a schematic representation of an exemplary IgG molecule with the various regions and sections labeled. The CDRs and framework regions (FR) of one of the two variable region light chains, and one of the two variable region heavy chains, are also labeled.

FIG. 2 shows the amino acid sequence of anti-CD160 antibody clone G6C9 antibody light chain variable region (SEQ ID NO:1). This figure shows the light chain CDRs boxed, which include CDRL1 (RASQSISDHLH; SEQ ID NO:2), CDRL2 (YASQSIS; SEQ ID NO:3), and CDRL3 (QHGHSFPFT, SEQ ID NO:4). FIG. 2 also shows Table 1 at the bottom, which shows exemplary amino acid substitutions that may be made in the CDRs of SEQ ID NO:1. Also, the murine light chain framework shown in SEQ ID NO:1, in some embodiments, it replaced with a suitable human or humanized light chain framework.

FIG. 3 shows the amino acid sequence of anti-CD160 antibody clone G6C9 antibody heavy chain variable region (SEQ ID NO:5). This figure shows the heavy chain CDRs boxed, which include CDRH1 (SDYYWS; SEQ ID NO:6), CDRH2 (YITYDGITNYSPSLKN; SEQ ID NO:7), and CDRH3 (DHYYNYFDY, SEQ ID NO:8). FIG. 3 also shows Table 2 at the bottom, which shows exemplary amino acid substitutions that may be made in the CDRs of SEQ ID NO:1. Also, the murine heavy chain framework shown in SEQ ID NO:5, in some embodiments, it replaced with a suitable human or humanized heavy chain framework.

FIG. 4 shows the amino acid sequence of anti-CD160 antibody clone 4C10 antibody light chain variable region (SEQ ID NO:9). This figure shows the light chain CDRs boxed, which include CDRL1 (RASQSISNNLH; SEQ ID NO:10), CDRL2 (YASQSIS; SEQ ID NO:11), and CDRL3 (HQSNSWPHT, SEQ ID NO:12). FIG. 4 also shows Table 3 at the bottom, which shows exemplary amino acid substitutions that may be made in the CDRs of SEQ ID NO:9. Also, the murine light chain framework shown in SEQ ID NO:9, in some embodiments, it replaced with a suitable human or humanized light chain framework.

FIG. 5 shows the amino acid sequence of anti-CD160 antibody clone 4C10 antibody heavy chain variable region (SEQ ID NO:13). This figure shows the heavy chain CDRs boxed, which include CDRH1 (YWMQ; SEQ ID NO:14), CDRH2 (AIYPGDGDSRYTQKFKG; SEQ ID NO:15), and CDRH3 (RLGGFDY, SEQ ID NO:16). FIG. 5 also shows Table 4 at the bottom, which shows exemplary amino acid substitutions that may be made in the CDRs of SEQ ID NO:13. Also, the murine heavy chain framework shown in SEQ ID NO:13, in some embodiments, it replaced with a suitable human or humanized heavy chain framework.

FIG. 6 shows the nucleic acid sequences of anti-CD160 clone G6C9 variable region heavy and light chains. FIG. 6A shows the heavy chain nucleic acid sequence of G6C9 (SEQ ID NO:17). FIG. 6B shows the light chain nucleic acid sequence of G6C9 (SEQ ID NO:18).

FIG. 7 shows the nucleic acid sequences of anti-CD160 clone 4C10 variable region heavy and light chains. FIG. 7A shows the heavy chain nucleic acid sequence of 4C10 (SEQ ID NO:19). FIG. 7B shows the light chain nucleic acid sequence of 4C10 (SEQ ID NO:20).

FIG. 8 shows normal human PBMCs were stained with chloroform labeled, NK cell characterizing, antibodies.

FIG. 9 shows results from testing supernatant from sub-cloning culture for antibody binding to NK cell in normal human PBMCs.

FIG. 10 provides graphs showing the effect of intravenous administration of anti-NK1.1 monoclonal antibodies on blood PBMCs in naïve mice.

FIG. 11 provides graphs showing the effect of intravenous administration of anti NK1.1 monoclonal antibodies on splenic PBMCs in naïve mice.

FIG. 12 provides graphs showing the in vitro effects of IFT-100 chimeric monoclonal antibody on NK cells.

FIG. 13 provides graphs showing the in vitro and in vivo effects of anti-human NK cell monoclonal IFT-100.

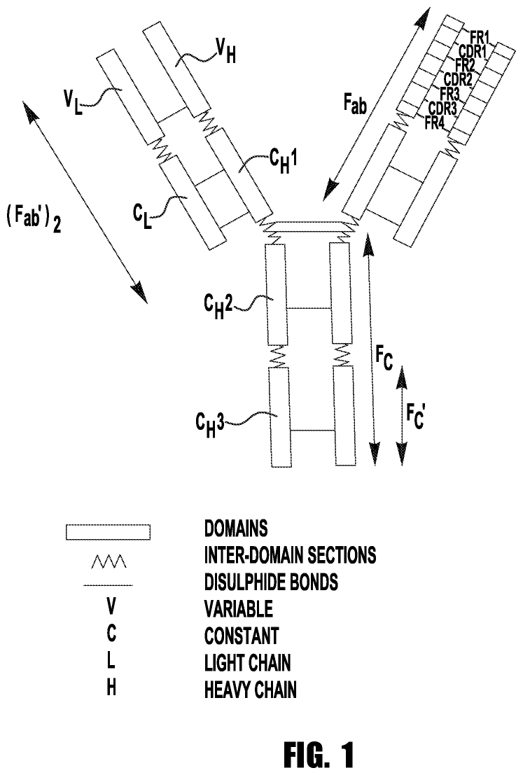

### DETAILED DESCRIPTION OF THE INVENTION

### Definitions

To facilitate an understanding of the invention, a number of terms are defined below.

The term “antibody,” as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each variable region (VH or VL) contains 3 CDRs, designated CDR1, CDR2 and CDR3. Each variable region also contains 4 framework sub-regions, designated FR1, FR2, FR3 and FR4.

As used herein, the term “antibody fragment or portion” refers to a portion of an intact antibody. Examples of antibody fragments or portions include, but are not limited to, linear antibodies, single-chain antibody molecules, Fv, Fab and F(ab′)2 fragments, and multi-specific antibodies formed from antibody fragments. The antibody fragments preferably retain at least part of the heavy and/or light chain variable region.

As used herein, the terms “complementarity determining region” and “CDR” refer to the regions that are primarily responsible for antigen-binding. There are three CDRs in a light chain variable region (CDRL1, CDRL2, and CDRL3), and three CDRs in a heavy chain variable region (CDRH1, CDRH2, and CDRH3). The residues that make up these six CDRs have been characterized by Kabat and Chothia as follows: residues 24-34 (CDRL1), 50-56 (CDRL2) and 89-97 (CDRL3) in the light chain variable region and 31-35 (CDRH1), 50-65 (CDRH2) and 95-102 (CDRH3) in the heavy chain variable region; Kabat et al., (1991) Sequences of Proteins of Immunological Interest, 5th Ed. Public Health Service, National Institutes of Health, Bethesda, MD., herein incorporated by reference; and residues 26-32 (CDRL1), 50-52 (CDRL2) and 91-96 (CDRL3) in the light chain variable region and 26-32 (CDRH1), 53-55 (CDRH2) and 96-101 (CDRH3) in the heavy chain variable region; Chothia and Lesk (1987) J. Mol. Biol. 196: 901-917, herein incorporated by reference. Unless otherwise specified, the terms “complementarity determining region” and “CDR” as used herein, include the residues that encompass both the Kabat and Chothia definitions (i.e., residues 24-34 (CDRL1), 50-56 (CDRL2), and 89-97 (CDRL3) in the light chain variable region; and 26-35 (CDRH1), 50-65 (CDRH2), and 95-102 (CDRH3)). Also, unless specified, as used herein, the numbering of CDR residues is according to Kabat.

As used herein, the term “framework” refers to the residues of the variable region other than the CDR residues as defined herein. There are four separate framework sub-regions that make up the framework: FR1, FR2, FR3, and FR4. In order to indicate if the framework sub-region is in the light or heavy chain variable region, an “L” or “H” may be added to the sub-region abbreviation (e.g., “FRL1” indicates framework sub-region 1 of the light chain variable region). Unless specified, the numbering of framework residues is according to Kabat. It is noted that, in certain embodiments, the CD160 binding molecules of the present invention may have less than a complete framework (e.g. the CD160 binding molecule may have a portion of a framework that only contains one or more of the four sub-regions).

As used herein, the term “fully human framework” means a framework with an amino acid sequence found naturally in humans. Examples of fully human frameworks, include, but are not limited to, KOL, NEWM, REI, EU, TUR, TEI, LAY and POM (See, e.g., Kabat et al., (1991) Sequences of Proteins of Immunological Interest, US Department of Health and Human Services, NIH, USA; and Wu et al., (1970) J. Exp. Med. 132, 211-250, both of which are herein incorporated by reference).

As used herein, the terms “subject” and “patient” refer to any animal, such as a mammal like a dog, cat, bird, livestock, and preferably a human.

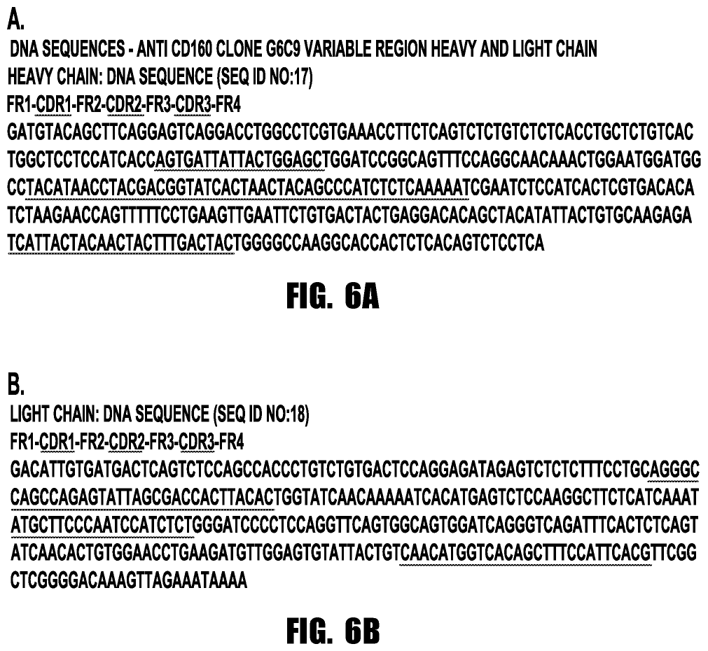

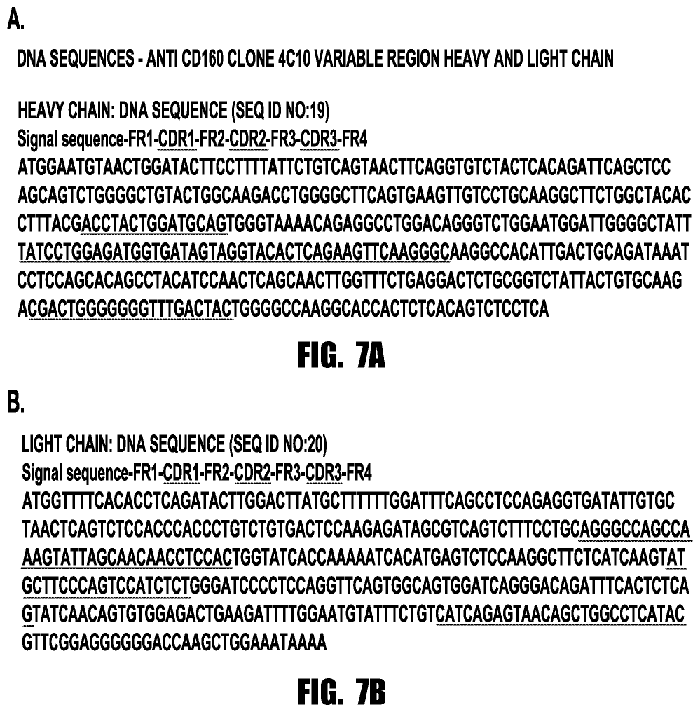

As used herein, the term “codon” or “triplet” refers to a group of three adjacent nucleotide monomers which specify one of the naturally occurring amino acids found in polypeptides. The term also includes codons which do not specify any amino acid. It is also noted that, due to the degeneracy of the genetic code, there are many codons that code for the same amino acid. As such, many of the bases of the nucleic acid sequences of the present invention (see, FIGS. 6 and 7, SEQ ID NOS:17-20) can be changed without changing the actual amino acid sequence that is encoded. The present disclosure is intended to encompass all such nucleic acid sequences.

As used herein, the terms “an oligonucleotide having a nucleotide sequence encoding a polypeptide,” “polynucleotide having a nucleotide sequence encoding a polypeptide,” and “nucleic acid sequence encoding a peptide” means a nucleic acid sequence comprising the coding region of a particular polypeptide. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements.

Also, as used herein, there is no size limit or size distinction between the terms “oligonucleotide” and “polynucleotide.” Both terms simply refer to molecules composed of nucleotides. Likewise, there is no size distinction between the terms “peptide” and “polypeptide.” Both terms simply refer to molecules composed of amino acid residues.

As used herein, the term “the complement of” a given sequence is used in reference to the sequence that is completely complementary to the sequence over its entire length. For example, the sequence 5′-A-G-T-A-3′ is “the complement” of the sequence 3′-T-C-A-T-5′. The present disclosure also provides the complement of the sequences described herein (e.g., the complement of the nucleic acid sequences in SEQ ID NOS: 17-20, and truncated versions thereof).

The term “isolated” when used in relation to a nucleic acid, as in “an isolated oligonucleotide” or “isolated polynucleotide” or “isolated nucleic acid sequence encoding an CD160 binding molecule” (see, e.g., FIGS. 6 and 7) refers to a nucleic acid sequence that is identified and separated from at least one contaminant nucleic acid with which it is ordinarily associated (e.g. host cell proteins).

As used herein, the term “purified” or “to purify” refers to the removal of contaminants from a sample. For example, CD160 binding molecules (e.g., antibodies or antibody fragments) may be purified by removal of contaminating non-immunoglobulin proteins; they are also purified by the removal of immunoglobulins that do not bind to the same antigen. The removal of non-immunoglobulin proteins and/or the removal of immunoglobulins that do not bind the particular antigen results in an increase in the percentage of antigen specific immunoglobulins in the sample. In another example, recombinant antigen-specific polypeptides are expressed in bacterial host cells and the polypeptides are purified by the removal of host cell proteins; the percentage of recombinant antigen-specific polypeptides is thereby increased in the sample.

As used herein, the term “Fc region” refers to a C-terminal region of an immunoglobulin heavy chain. The “Fc region” may be a native sequence Fc region or a variant Fc region (e.g., with increased or decreased effector functions).

As used herein, the terms “cardiovascular disease” (CVD) or “cardiovascular disorder” are terms used to classify numerous conditions affecting the heart, heart valves, and vasculature (e.g., veins and arteries) of the body and encompasses diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

As used herein, the term “atherosclerotic cardiovascular disease” or “disorder” refers to a subset of cardiovascular disease that include atherosclerosis as a component or precursor to the particular type of cardiovascular disease and includes, without limitation, CAD, PAD, cerebrovascular disease. Atherosclerosis is a chronic inflammatory response that occurs in the walls of arterial blood vessels. It involves the formation of atheromatous plaques that can lead to narrowing (“stenosis”) of the artery, and can eventually lead to partial or complete closure of the arterial opening and/or plaque ruptures. Thus, atherosclerotic diseases or disorders include the consequences of atheromatous plaque formation including, without limitation, stenosis or narrowing of arteries, rupture of the plaque that may lead to ischemic events such as myocardial infarction and stroke, heart failure, aneurysm formation including aortic aneurysm, and aortic dissection.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

A “prophylactic” or “preventive” treatment is a treatment administered to a subject who does not exhibit signs of a disease or disorder, or exhibits only early signs of the disease or disorder, for the purpose of decreasing the risk of developing pathology associated with the disease or disorder. Preventative treatment may be appropriate for treating subjects having an increased risk of developing a disease, such as a disease associated with inflammation.

The present invention relates to CD160 binding molecules and nucleic acid sequences encoding such molecules. In particular embodiments, the present invention provides CD160 binding molecules (e.g., monoclonal antibodies or antibody fragments) with particular light chain and/or heavy chain CDRs (e.g., selected from SEQ ID NOS:2-4, 10-12, 6-8, and 14-16) and methods for using such molecules to treat an inflammatory disease, cancer, a chronic infection, and a refractory infection, or to determine whether cells with the CD160 surface receptor are present in certain tissues or cells being examined

In certain embodiments the CD160 binding molecules herein are used to inhibit inflammation and thereby prevent progressive myocardial dysfunction and heart failure in AMI, in cardiogenic shock, and in cardiomyopathy, as well as improves outcomes in auto-immune and auto-inflammatory diseases. In certain embodiments, such CD160 binding molecules are used to treat inflammation. In certain embodiments, such CD160 binding molecules are used to determine whether cells with the CD160 surface receptor are present in certain tissues or cells being examined.

The CD160 protein (sometimes referred to as the CD160 antigen, or the CD160 surface receptor) is encoded by the CD160 gene. The CD160 protein is a 27 kDa glycoprotein whose expression is tightly associated with peripheral blood NK cells and CD8 T lymphocytes with cytolytic effector activity. The cDNA sequence of CD160 predicts a cysteine-rich, glycosylphosphatidylinositol-anchored protein of 181 amino acids. CD160 is expressed at the cell surface as a tightly disulfide-linked multimer.

The CD160 binding molecules described herein have an effect on the levels and/or activity of natural killer (NK) cells. NK cells are a type of cytotoxic lymphocyte that play in important role in the innate immune system. In some embodiments, the CD160 binding molecules described herein are used in methods for reducing, alleviating and/or preventing the kind of excessive, persistent inflammation associated with a variety of diseases. The CD160 binding molecules can be used to deplete and/or inhibit NK cells, thereby decreasing inflammation. In other embodiments, the CD160 binding molecules can be used to activate NK cells and enhance inflammation, and used to treat infection, cancer, and other diseases in which inflammation can exert beneficial effects.

An excessive and persistent inflammatory response contributes to the progressive deterioration of myocardial function seen both in heart attacks, in cardiogenic shock, and in heart failure (as well as in rheumatoid arthritis, inflammatory bowel disease, psoriasis, septic shock, etc.). Therefore, inhibition of inflammation is used to improve outcomes of these diseases by employing a CD160 binding molecule.

In certain embodiments, the CD160 binding molecules comprise one or more of the CDRs shown in SEQ ID NOS:2-4, 6-8, 10-12, and 14-16 and/or CDRs with one or more conservative or non-conservative amino acid changes in these SEQ ID NOS. Also provided are nucleic acid sequences substantially similar to SEQ ID NOS: 17-20 (e.g., sequences with at least 80 . . . 90 . . . or 99% sequence identity). Changes to the amino acid sequences of the CDRs may be generated by changing the nucleic acid sequence encoding the amino acid sequence. A nucleic acid sequence encoding a variant of a given CDR may be prepared by methods known in the art using the guidance of the present specification for particular sequences. These methods include, but are not limited to, preparation by site-directed (or oligonucleotide-mediated) mutagenesis, PCR mutagenesis, and cassette mutagenesis of an earlier prepared nucleic acid encoding the CDR.

Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the starting CDR (see, e.g., Vallette et. al., (1989) Nucleic Acids Res. 17: 723-733, hereby incorporated by reference). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., (1985) Gene 34: 315-323, hereby incorporated by reference. The starting material is the plasmid (or other vector) comprising the starting CDR DNA to be mutated. The codon(s) in the starting DNA to be mutated are identified. There should be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a CDR variant can be determined, and a nucleic acid sequence encoding such amino acid sequence variant can be generated synthetically. Conservative modifications in the amino acid sequences of the CDRs may also be made. Naturally occurring residues are divided into classes based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.
   Conservative substitutions will entail exchanging a member of one of these classes for another member of the same class in a particular CDR, such as in SEQ ID NOS:2-4, 6-8, 10-12, and 14-16. The present invention also provides the complement of the nucleic acid sequences shown SEQ ID NOS: 17-20, as well as nucleic acid sequences that will hybridize to these nucleic acid sequences under low, medium, and high stringency conditions.

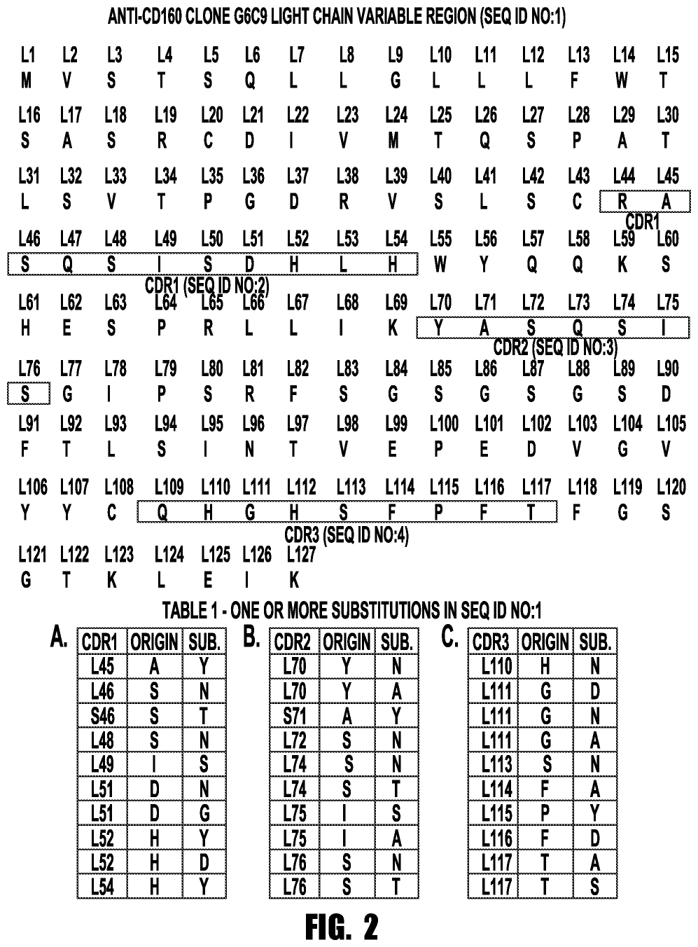

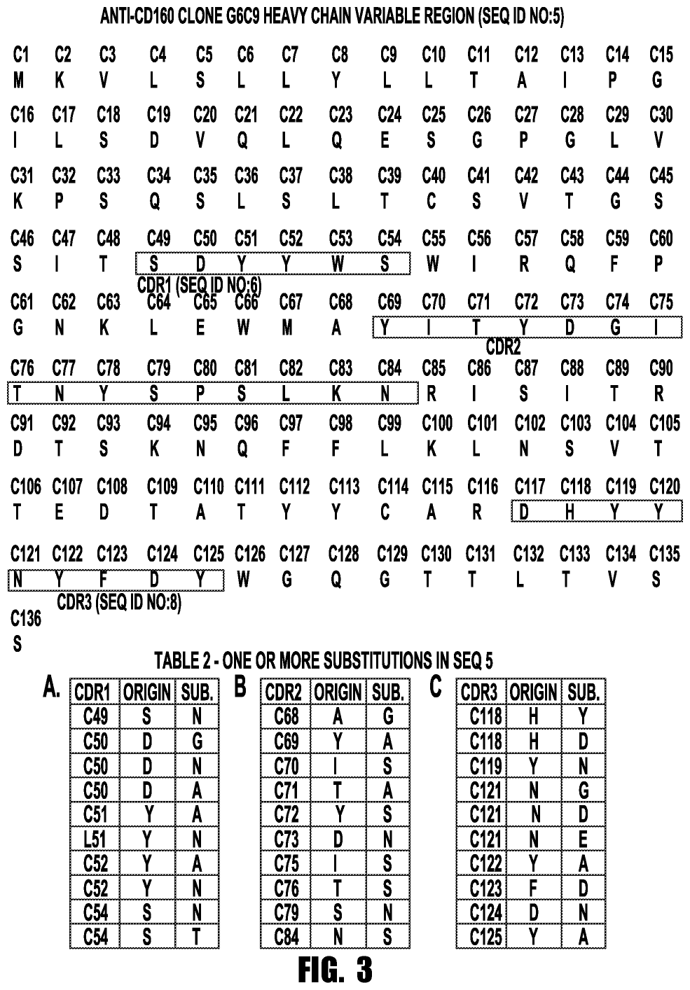

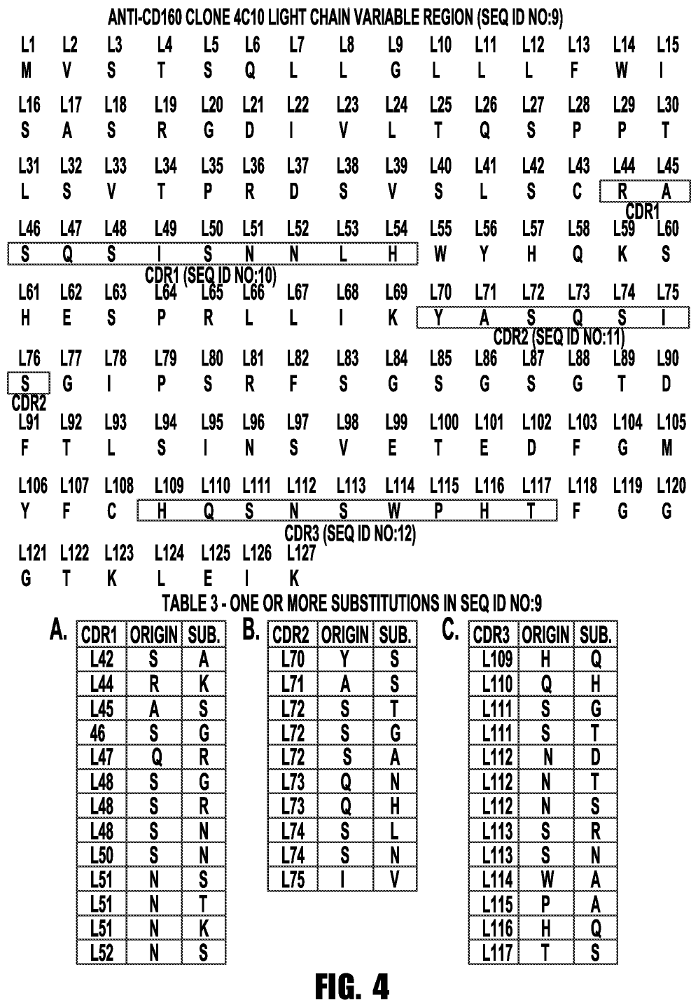

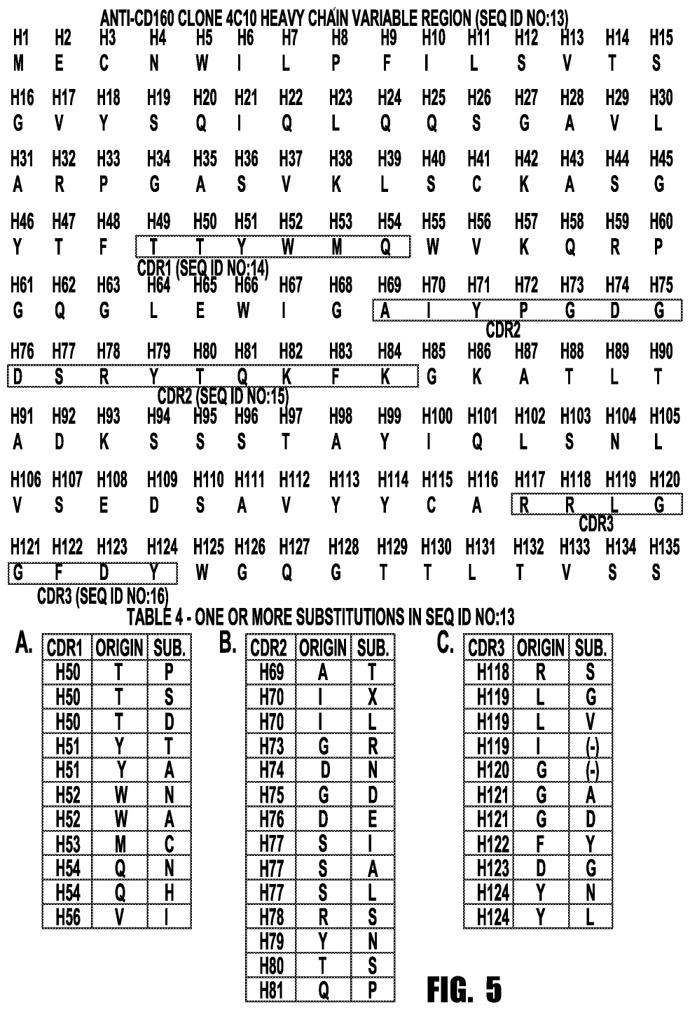

The CDRs of the present invention may be employed with any type of suitable framework. The frameworks shown in FIGS. 2-3 are the original mouse frameworks from the G6C9 antibody, and the frameworks shown in FIGS. 4-5 are the original mouse frameworks for the 4C10 antibody. The CDRs may be used with other murine frameworks, which are known in the art. In other embodiments, the CDRs are used with fully human frameworks, or framework sub-regions. For example, the NCBI web site contains the sequences for known human framework regions. Examples of human VH sequences include, but are not limited to, VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81, which are provided in Matsuda et al., (1998) J. Exp. Med. 188:1973-1975, that includes the complete nucleotide sequence of the human immunoglobulin chain variable region locus, herein incorporated by reference.

Examples of human VK sequences include, but are not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8, which are provided in Kawasaki et al., (2001) Eur. J. Immunol. 31:1017-1028; Schable and Zachau, (1993) Biol. Chem. Hoppe Seyler 374:1001-1022; and Brensing-Kuppers et al., (1997) Gene 191:173-181, all of which are herein incorporated by reference. Examples of human VL sequences include, but are not limited to, V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4- 3, V4-4, V4-6, V5-1, V5-2, V5-4, and V5-6, which are provided in Kawasaki et al., (1997) Genome Res. 7:250-261, herein incorporated by reference. Fully human frameworks can be selected from any of these functional germline genes. Generally, these frameworks differ from each other by a limited number of amino acid changes. These frameworks may be used with the CDRs described herein. Additional examples of human frameworks which may be used with the CDRs of the present invention include, but are not limited to, KOL, NEWM, REI, EU, TUR, TEI, LAY and POM (See, e.g., Kabat et al., (1991) Sequences of Proteins of Immunological Interest, US Department of Health and Human Services, NIH, USA; and Wu et al., (1970), J. Exp. Med. 132:211-250, both of which are herein incorporated by reference).

In certain embodiments, the CD160 binding molecules of the present invention comprise antibodies or antibody fragments (e.g., comprising one or more of the CDRs described herein). An antibody, or antibody fragment, of the present invention can be prepared, for example, by recombinant expression of immunoglobulin light and heavy chain genes in a host cell. For example, to express an antibody recombinantly, a host cell may be transfected with one or more recombinant expression vectors carrying DNA fragments encoding the immunoglobulin light and heavy chains of the antibody such that the light and heavy chains are expressed in the host cell and, preferably, secreted into the medium in which the host cell is cultured, from which medium the antibody can be recovered. Standard recombinant DNA methodologies may be used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al., all of which are herein incorporated by reference.

In certain antibodies, the anti-CD160 antibodies prepared herein have an IgG isotype constant regions as shown in Tables 5 and 6 below.

TABLE 5

| IgG Isotype | |
|---|---|
| Constant region | Accetion # |
| IgG Light Kappa | P0DOX7.1 |
| IgG Light Lambda | P0DOX8.1 |
| IgG1 Heavy | P01857 |
| IgG2 Heavy | P01859 |
| IgG3 Heavy | P01860 |
| IgG4 Heavy | P01861 |

TABLE 6
Mouse

| IgG Isotype | |
|---|---|
| Constant | |
| region | Accession # |
| IgG Light | BAR42292, |
| Kappa | AWY10547.1, AAH80787.1 |
| IgG Light | AAO53381.1, |
| Lambda | QCF41906.1, |
| | AAO53357.1 |
| IgG1 Heavy | CZF87189.1, |
| | AAB06744.1, |
| | CAD68983.1 |
| IgG2a Heavy | AGH20709.1, BAC44883.1, |
| | AAI08376.1, |
| IgG2b Heavy | CZF87191.1, |
| | ACX70084.1, |

To express an antibody with one or more of the CDRs of the present invention, DNA fragments encoding the light and heavy chain variable regions are first obtained. These DNAs can be obtained by amplification and modification of germline light and heavy chain variable sequences using the polymerase chain reaction (PCR).

Once the germline VH and VL fragments are obtained, these sequences can be mutated to encode one or more of the CDR amino acid sequences disclosed herein (see, e.g., SEQ ID NOS:2-4, 6-8, 10-12, and 14-16). The amino acid sequences encoded by the germline VH and VL DNA sequences may be compared to the CDRs sequence(s) desired to identify amino acid residues that differ from the germline sequences. Then the appropriate nucleotides of the germline DNA sequences are mutated such that the mutated germline sequence encodes the selected CDRs (e.g., the six CDRs shown in FIG. 2-3 or 4-5), using the genetic code to determine which nucleotide changes should be made. Mutagenesis of the germline sequences may be carried out by standard methods, such as PCR-mediated mutagenesis (in which the mutated nucleotides are incorporated into the PCR primers such that the PCR product contains the mutations) or site-directed mutagenesis. In other embodiments, the variable region is synthesized de novo (e.g., using a nucleic acid synthesizer).

Once DNA fragments encoding the desired VH and VL segments are obtained (e.g., by amplification and mutagenesis of germline VH and VL genes, or synthetic synthesis, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operably linked to another DNA fragment encoding another polypeptide, such as an antibody constant region or a flexible linker. The isolated DNA encoding the VH region can be converted to a full-length heavy chain gene by operably linking the VH-encoding DNA to another DNA molecule encoding heavy chain constant regions (CH1, CH2 and CH3). The sequences of mouse and human heavy chain constant region genes are known in the art and DNA fragments encompassing these regions can be obtained by standard PCR amplification. The heavy chain constant region can be, for example, an IgG1, IgG2, IgG3, IgG4, IgA, IgE, IgM or IgD constant region. For a Fab fragment heavy chain gene, the VH-encoding DNA can be operably linked to another DNA molecule encoding only the heavy chain CH1 constant region.

The isolated DNA encoding the VL region can be converted to a full-length light chain gene (as well as a Fab light chain gene) by operably linking the VL-encoding DNA to another DNA molecule encoding the light chain constant region, CL. The sequences of mouse and human light chain constant region genes are known in the art (see e.g., Kabat, E. A., et al., (1991) Sequences of Proteins of immunological Interest, Fifth Edition, U.S. Department of Health and Human Services. NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments may be operably linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (see e.g., Huston et al., (1988) Proc. Natl. Acad. Sci. USA 85:5879-5883; and McCafferty et al., (1990) Nature 348:552-554), all of which are herein incorporated by reference).

To express the antibodies, or antibody fragments of the invention, DNAs encoding partial or full-length light and heavy chains, (e.g. obtained as described above), may be inserted into expression vectors such that the genes are operably linked to transcriptional and translational control sequences. In this context, the term “operably linked” is intended to mean that an antibody gene is ligated into a vector such that transcriptional and translational control sequences within the vector serve their intended function of regulating the transcription and translation of the antibody gene. The expression vector and expression control sequences are generally chosen to be compatible with the expression host cell used. The antibody light chain gene and the antibody heavy chain gene can be inserted into separate vectors or, more typically, both genes are inserted into the same expression vector. The antibody genes may be inserted into the expression vector by standard methods (e.g., ligation of complementary restriction sites on the antibody gene fragment and vector, or blunt end ligation if no restriction sites are present). Prior to insertion of the light or heavy chain sequences, the expression vector may already carry antibody constant region sequences. For example, one approach to converting the VH and VL sequences to full-length antibody genes is to insert them into expression vectors already encoding heavy chain constant and light chain constant regions, respectively, such that the VH segment is operably linked to the CH segment(s) within the vector and the VL segment is operably linked to the CL segment within the vector. Additionally, or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention may carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990), herein incorporated by reference. It will be appreciated by those skilled in the art that the design of the expression vector, including the selection of regulatory sequences may depend on such factors as the choice of the host cell to be transformed, the level of expression of protein desired, etc. In certain embodiments, regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma virus. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., all of which are herein incorporated by reference.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634.665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr- host cells with methotrexate selection/amplification) and the neomycin gene (for G418 selection).

For expression of the light and heavy chains, the expression vector(s) encoding the heavy and light chains may be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

In certain embodiments, the expression vector used to express the CD160 binding molecules of the present invention are viral vectors, such as retro-viral vectors. Such viral vectors may be employed to generate stably transduced cell lines (e.g. for a continues source of the CD160 binding molecules). In some embodiments, the GPEX gene product expression technology (from Gala Design, Inc., Middleton, Wis.) is employed to generate CD160 binding molecules (and stable cell lines expressing the CD160 binding molecules). In particular embodiments, the expression technology described in WO0202783 and WO0202738 to Bleck et al. (both of which are herein incorporated by reference in their entireties) is employed.

Mammalian host cells for expressing the recombinant antibodies of the invention include PER.C6™ cells (Crucell, The Netherlands), Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In other embodiments, the host cells express GnT III as described in WO9954342 and U.S. Pat. Pub. 20030003097, both herein incorporated by reference, such that expressed CD160 binding molecules have increased ADCC activity. When recombinant expression vectors encoding antibody genes are introduced into mammalian host cells, the antibodies are generally produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present invention. For example, it may be desirable to transfect a host cell with DNA encoding either the light chain or the heavy chain of an antibody of this invention. Recombinant DNA technology may also be used to remove some or all of the DNA encoding either or both of the light and heavy chains that is not necessary for binding to CD160s. The molecules expressed from such truncated DNA molecules are also encompassed by the antibodies of the invention. In addition, bi-functional antibodies may be produced in which one heavy and one light chain are an antibody of the invention and the other heavy and light chain are specific for an antigen other than an CD160 (e.g., by crosslinking an antibody of the invention to a second antibody by standard chemical crosslinking methods).

In certain embodiments, the antibodies and antibody fragments of the present invention are produced in transgenic animals. For example, transgenic sheep and cows may be engineered to produce the antibodies or antibody fragments in their milk (see, e.g., Pollock D P, et al., (1999) Transgenic milk as a method for the production of recombinant antibodies. J. Immunol. Methods 231:147-157, herein incorporated by reference). The antibodies and antibody fragments of the present invention may also be produced in plants (see, e.g., Larrick et al., (2001) Production of secretory IgA antibodies in plants. Biomol. Eng. 18:87-94, herein incorporated by reference). Additional methodologies and purification protocols are provided in Humphreys et al., (2001) Therapeutic antibody production technologies: molecules applications, expression and purification, Curr. Opin. Drug Discov. Devel. 4:172-185, herein incorporated by reference. In certain embodiments, the antibodies or antibody fragments of the present invention are produced by transgenic chickens (see, e.g., US Pat. Pub. Nos. 20020108132 and 20020028488, both of which are herein incorporated by reference).

The CD160 binding proteins described herein are designed for specific binding, as a result of the affinity of complementary determining region of the antibody for an epitope on CD160. An antibody “specifically binds” when the antibody preferentially binds a target structure, or subunit thereof, but binds to a substantially lesser degree or does not bind to a biological molecule that is not a target structure. In some embodiments, the antibody specifically binds to the CD160 with a specific affinity of between 10⁻⁸ M and 10⁻¹¹ M. In some embodiments, an antibody or antibody fragment binds to the target analyte with a specific affinity of greater than 10⁻⁷ M, 10⁻⁸ M, 10⁻⁹ M, 10⁻¹⁰ M, or 10⁻¹¹ M, between 10⁻⁸ M-10⁻¹¹ M, 10⁻⁹ M-10⁻¹⁰ M, and 10⁻¹⁰ M -10⁻¹¹ M. In a preferred aspect, specific activity is measured using a competitive binding assay as set forth in Ausubel FM, (1994). Current Protocols in Molecular Biology. Chichester: John Wiley and Sons (“Ausubel”), which is incorporated herein by reference.

In certain embodiments, the CD160 binding molecules of the present invention (e.g., as antibodies or antibody fragments) are useful for immunoassays which detect or quantify CD160 in a sample (e.g., a purified blood sample from a subject). In some embodiments, an immunoassay for CD160 typically comprises incubating a biological sample in the presence of a detectably labeled antibody or antibody fragment of the present invention capable of selectively binding to CD160, and detecting the labeled peptide or antibody which is bound in a sample. Various clinical assay procedures are well known in the art.

The present disclosure provides immunoassay methods for determining the presence, amount or concentration of CD160 in a test sample. Any suitable assay known in the art can be used in such a method. Examples of such assays include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), an ARCHITECT assay (ABBOTT), a bioluminescence resonance energy transfer (BRET), and homogeneous chemiluminescent assay, etc.

A CD160 binding molecule can be captured on beads or nitrocellulose, or on any other solid support which is capable of immobilizing soluble proteins (e.g., magnetic beads). A CD160 containing sample is then added to the support which is subsequently washed with suitable buffers to remove unbound proteins. A second, detectably labeled, molecule (e.g., antibody or peptide) that can bind to the CD160 binding molecule is added to the solid phase support that can then be washed with the buffer a second time to remove unbound molecules. The amount of bound label on the solid support can then be detected by known methods.

Detectably labeling a CD160 binding molecule can be accomplished by coupling to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label the CD160 binding molecules of the present invention include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

In some embodiments of the present invention, the CD160 which is detected by the above assays can be present in a biological sample. Any sample containing CD160s can be used. Preferably, the sample is a biological fluid such as, for example, blood, serum, lymph, urine, cerebrospinal fluid, amniotic fluid, synovial fluid, a tissue extract or homogenate, and the like. However, the invention is not limited to assays using only these samples, as it is possible for one of ordinary skill in the art to determine suitable conditions which allow the use of other samples.

In situ detection can be accomplished by removing a histological specimen from a patient, and providing the combination of labeled CD160 binding molecules of the present invention to such a specimen. The CD160 binding molecule is preferably provided by applying or by overlaying the labeled CD160 binding molecule to a biological sample. Through the use of such a procedure, it is possible to determine not only the presence of CD160s, but also the distribution of CD160 in the examined tissue.

In certain embodiments, provided here are kits for the detection of CD160 that include a CD160 detection molecule. Such kits may include any of the immunodiagnostic reagents described herein and may further include instructions for the use of the immunodiagnostic reagents in immunoassays for determining the presence of CD160 in a test sample. The kits may also include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit may be provided in suitable containers as necessary, e.g., a microtiter plate. The kit can further include containers for holding or storing a sample (e.g., a container or cartridge for a sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

Examples have been included to more clearly describe particular embodiments of the invention. However, there are a wide variety of other embodiments within the scope of the present invention, which should not be limited to the particular examples provided herein.

### EXAMPLES

### Example 1: Anti-CD160 Antibody Identification and Characterization

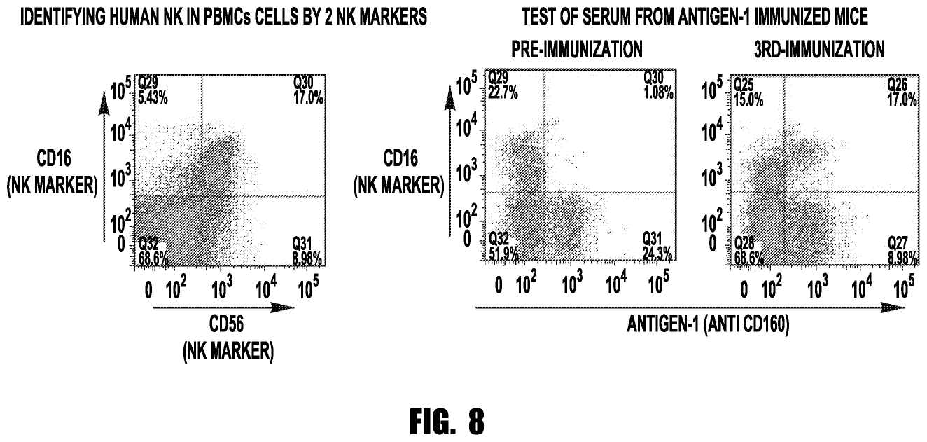

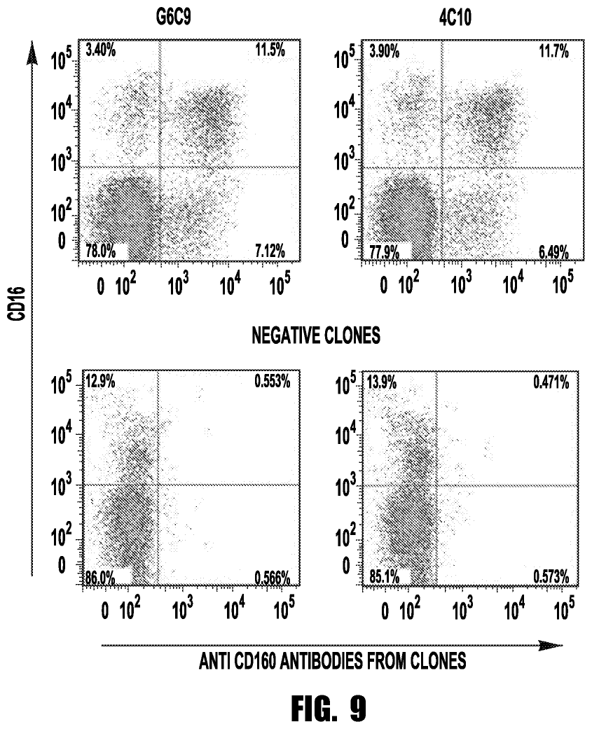

This example describes identifying and testing CD160 antibodies. The results of this Example are shown in FIGS. 8 and 9, and Table 7.

Validation of NK specific antibodies development in CD160 immunized mice was as follows. Mice were immunized with CD160 protein and serum samples were tested for human NK cell binding. Serum from pre-immunized mice and post 3rd immunization were incubated with normal human PBMCs. Thereafter cells were washed and incubated with a PE labeled secondary antibody (goat anti mouse IgG). Then cells were washed and stained with NK and other, cell type characterizing, antibodies. Results are shown in FIG. 8 as flow cytometry analysis of live cells. Results demonstrate co-staining of serum anti CD160 antibodies together with other NK cell specific markers.

Binding analysis of antibodies secreted from hybridoma cells, generated from CD160 immunized mice, was conducted as follows. Supernatant from hybridoma clones culture were analyzed by ELISA using CD160 antigen as target. Positive clones were selected based on high ELISA score. All clones were found negative for His-Tag that was included in the immunized antigen. Results are shown in Table 7.

TABLE 7
ELISA Binding analysis of Clones' Antibodies

| | | Binding score | Binding score |
|---|---|---|---|
| | Clone | to CD160 | to His-tag |
| Positive clones | 4C10 | 2.1407 | 0.085 |
| | G6C9 | 2.0142 | 0.056 |
| | C9C9 | 2.1362 | 0.072 |
| | A1A1 | 2.2587 | 0.075 |
| Negative clones | H6H5 | 1.9533 | 0.059 |
| | D1C7 | 1.9505 | 0.047 |
| | A8C9 | 1.9267 | 0.050 |
| | 6F12 | 1.9133 | 0.061 |
| | F3C9 | 1.908 | 0.071 |
| | 10H8 | 1.896 | 0.051 |
| | B3B10 | 1.8862 | 0.057 |
| | 11C9 | 1.8731 | 0.051 |
| | 12F2 | 1.8542 | 0.063 |

Binding confirmation following sub-cloning was conducted as follows. Positive clones tested by flow cytometry further went through a sub-cloning process. Supernatant from sub-cloning culture was tested for antibody binding to NK cell in normal human PBMCs. The staining procedure and flow cytometry analysis is as shown in FIG. 9. Negative clones were tested as control.

### Example 2: NK Cell Depletion Using Commercially-Available Anti NK1.1 Monoclonal Antibody in Mice

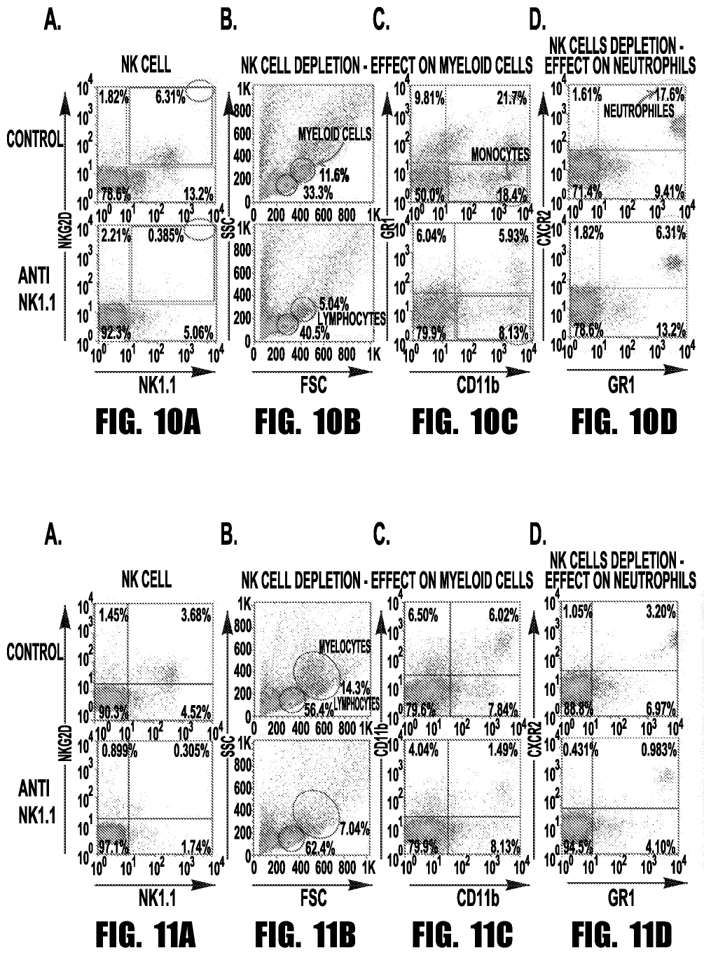

The effects of NK cell depletion (anti NK1.1 (clone PK136), Biolegend, Calif.) on peripheral blood mononuclear cells (PBMCs) on day 8 post-NK cell depletion are shown in FIG. 10. FIG. 10A shows the effect on NK cell population, FIG. 10B shows the effect on the proportion of circulating myeloid cells, and FIGS. 10C and 10D show the effect on monocyte and neutrophil populations, respectively. All indicated cell populations are pointed by arrows. The figures show that intravenous administration of commercially available anti-NK1.1 monoclonal antibody (mAb) results in several changes in blood PBMCs in naïve mice at 8 days post-administration. These changes include a reduction in NK cells, a reduction in the proportion of circulating myeloid cells, and reduction in both monocyte and neutrophil cell populations. These results demonstrate that NK cell depletion reduced circulating myeloid cells, which include both monocytes and neutrophils. This suggests that the impact of NK cell depletion is not limited to a direct effect on a target organ, but rather has the potential to exert major systemic effects.

FIG. 11 shows the effects of NK cell depletion on splenic PBMCs in naïve mice on day 6 after NK cell depletion. FIG. 11A shows the effect on NK cell population, FIG. 11B shows the effect on the proportion of myeloid cells, and FIGS. 11C and 11D show the effects on monocyte and neutrophil cell populations, respectively. The figures show that the intravenous administration of commercially available anti-NK1.1 mAb results in several changes in splenic PBMCs in naïve mice. These changes include a reduction in splenic NK cells, a reduction in the proportion of splenic myeloid cells, and reduction of both the monocyte and neutrophil cell populations. These results demonstrate that NK cell depletion reduces splenic myeloid cells, which include both monocytes and neutrophils. This further confirms that the impact of NK cell depletion is not limited to a direct effect on a target organ, but has the potential to exert major systemic effects.

### Example 3: In Vitro Effects of IFT-100 Chimeric mAb on NK Cells

The in vitro effects of IFT-100 chimeric mAbs on NK cells are shown in FIG. 12. The IFT-100 chimeric mAbs comprise a human IgG1 Fc region together with the human IFT-100 variable region. The chimeric IFT-100 antibody was incubated with human PBMCs, with or without added human serum, demonstrating complement dependent NK cell depletion. The results show that IFT-100 binds avidly to human NK cells and selectively depletes >70% of these cells in vitro.

### Example 4: In Vivo Effects of IFT-100 mAb

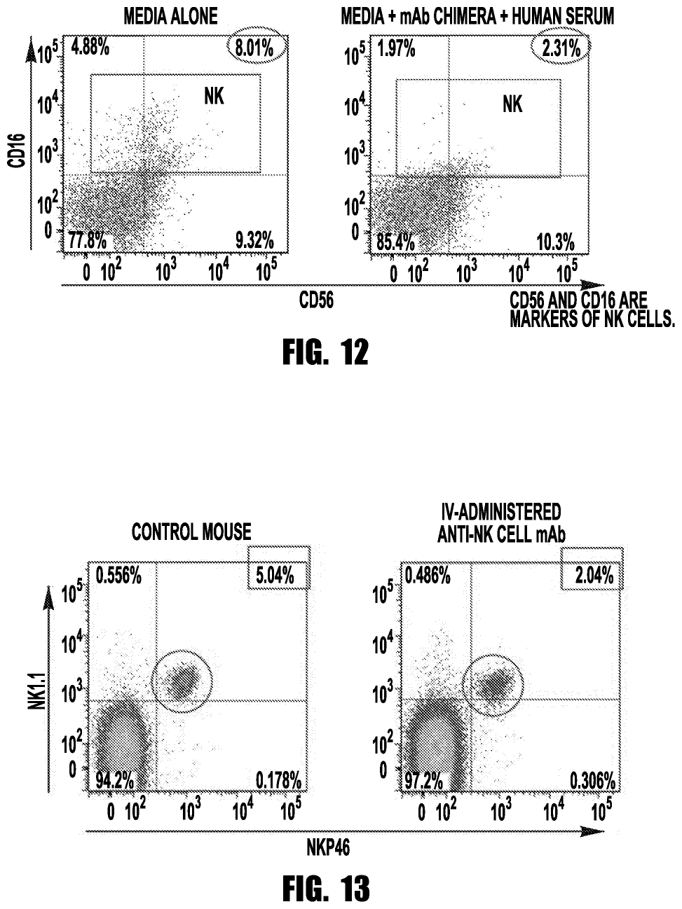

The in vivo effects of IFT-100 mAb are shown in FIG. 13. IFT-100 mAb (extracted from mouse hybridoma) was injected intravenously into naïve mice, and splenocytes and blood PBMCs were analyzed 48 hours after injection by flow cytometry. The results demonstrate that IFT-100 depleted about 60% of mouse NK cells in vivo.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A composition comprising a CD160 binding molecule, wherein said CD160 binding molecule comprises:
a) a light chain variable region, wherein said light chain variable region comprises;
i) a CDRL1 amino acid sequence selected from the group consisting of SEQ ID NO:2, SEQ ID NO:10, SEQ ID NO:2 with one or more substitutions from Table 1A and/or with one or more conservative amino acid changes, and SEQ ID NO:10 with one or more substitutions from Table 3A and/or one or more conservative amino acid changes;
ii) a CDRL2 amino acid sequence selected from the group consisting of SEQ ID NO:3, SEQ ID NO:11, SEQ ID NO:3 with one or more substitutions from Table 1B and/or with one or more conservative amino acid changes, and SEQ ID NO:11 with one or more substitutions from Table 3B and/or one or more conservative amino acid changes; and
iii) a CDRL3 amino acid sequence selected from the group consisting of SEQ ID NO:4, SEQ ID NO:12, SEQ ID NO:4 with one or more substitutions from Table 1C and/or with one or more conservative amino acid changes, and SEQ ID NO:12 with one or more substitutions from Table 3C and/or one or more conservative amino acid changes; and
b) a heavy chain variable region, wherein said heavy chain variable region comprises:
i) a CDRH1 amino acid sequence selected from the group consisting of SEQ ID NO:6, SEQ ID NO:14, SEQ ID NO:6 with one or more substitutions from Table 2A and/or with one or more conservative amino acid changes, and SEQ ID NO:14 with one or more substitutions from Table 4A and/or with one or more conservative amino acid changes;
ii) a CDRH2 amino acid sequence selected from the group consisting of SEQ ID NO:7, SEQ ID NO:15, SEQ ID NO:7 with one or more substitutions from Table 2B and/or with one or more conservative amino acid changes, and SEQ ID NO:15 with one or more substitutions from Table 4B and/or with one or more conservative amino acid changes; and
iii) a CDRH3 amino acid sequence selected from the group consisting of SEQ ID NO:8, SEQ ID NO:16, SEQ ID NO:8 with one or more substitutions from Table 2C and/or with one or more conservative amino acid changes, and SEQ ID NO:16 with one or more substitutions from Table 4C and/or with one or more conservative amino acid changes.

2. The composition of claim 1, wherein said CD160 binding molecule is an antibody, minibody, diabody, scFv, or antibody fragment capable of binding human CD160.

3. The composition of claim 2, wherein said antibody fragment is a Fab or Fv antibody fragment.

4. The composition of claim 2, wherein said antibody fragment comprises an antigen binding portion of the G6C9 or 4C10 antibody.

5. The composition of claim 1, wherein said light and/or heavy chain variable region comprises a mouse or human framework region.

6. The composition of claim 1, wherein said CD160 binding molecule further comprises a light chain constant region and a CH1 heavy chain constant region.

7. The composition of claim 6, wherein said CD160 binding molecule further comprises a CH2 heavy chain constant region and/or a CH3 heavy chain constant region.

8. The composition of claim 1, wherein said CD160 binding molecule is an antibody or antigen binding portion thereof that has an Fc region characterized in that it: i) has an Fc cellular binding site; ii) has a Fc complement binding site; and/or 3) has a linked toxin that is internalized into a cell.

9. The composition of claim 8, wherein said light chain constant region is human or a humanized murine, and/or wherein said CH1, CH2, and CH3 heavy chain constant regions are human or are humanized murine.

10. The composition of claim 1, wherein said CD160 binding molecule comprises an antibody, wherein the light chain constant region of said antibody is selected from: IgG Kappa and IgG Lambda, and wherein the heavy chain constant region of said antibody is selected from: IgG1, IgG2, IgG3, and IgG4.

11. The composition of claim 1, wherein said CD160 binding molecule comprises an antibody, or antigen binding portion thereof, which is fucosylated or non-fucosylated.

12. The composition of claim 1, further comprising a physiologically tolerable buffer.

13. A composition comprising at least one of the following:
a) a first nucleic acid sequence encoding a light chain variable region, wherein said light chain variable region comprises:
i) a CDRL1 amino acid sequence selected from the group consisting of SEQ ID NO:2, SEQ ID NO:10, SEQ ID NO:2 with one or more substitutions from Table 1A and/or with one or more conservative amino acid changes, and SEQ ID NO:10 with one or more substitutions from Table 3A and/or one or more conservative amino acid changes;
ii) a CDRL2 amino acid sequence selected from the group consisting of SEQ ID NO:3, SEQ ID NO:11, SEQ ID NO:3 with one or more substitutions from Table 1B and/or with one or more conservative amino acid changes, and SEQ ID NO:11 with one or more substitutions from Table 3B and/or one or more conservative amino acid changes; and
iii) a CDRL3 amino acid sequence selected from the group consisting of SEQ ID NO:4, SEQ ID NO:12, SEQ ID NO:4 with one or more substitutions from Table 1C and/or with one or more conservative amino acid changes, and SEQ ID NO:12 with one or more substitutions from Table 3C and/or one or more conservative amino acid changes; and/or
b) a second nucleic acid sequence encoding a heavy chain variable region, wherein said heavy chain variable region comprises:
i) a CDRH1 amino acid sequence selected from the group consisting of SEQ ID NO:6, SEQ ID NO:14, SEQ ID NO:6 with one or more substitutions from Table 2A and/or with one or more conservative amino acid changes, and SEQ ID NO:14 with one or more substitutions from Table 4A and/or with one or more conservative amino acid changes;
ii) a CDRH2 amino acid sequence selected from the group consisting of SEQ ID NO:7, SEQ ID NO:15, SEQ ID NO:7 with one or more substitutions from Table 2B and/or with one or more conservative amino acid changes, and SEQ ID NO:15 with one or more substitutions from Table 4B and/or with one or more conservative amino acid changes; and
iii) a CDRH3 amino acid sequence selected from the group consisting of SEQ ID NO:8, SEQ ID NO:16, SEQ ID NO:8 with one or more substitutions from Table 2C and/or with one or more conservative amino acid changes, and SEQ ID NO:16 with one or more substitutions from Table 4C and/or with one or more conservative amino acid changes.

14. The composition of claim 13, further comprising an expression vector, and wherein said first and/or second nucleic acid sequences are present in said expression vector.

15. The composition of claim 13, wherein said light and heavy chain variable regions are part of a CD160 binding molecule.

16. The composition of claim 15, wherein said CD160 binding molecule is an antibody, minibody, diabody, scFv, or antibody fragment capable of binding human CD160.

17. The composition of claim 16, wherein said antibody fragment is a Fab or Fv antibody fragment.

18. The composition of claim 16, wherein said antibody fragment comprises an antigen binding portion of the G6C9 or 4C10 antibody.

19. The composition of claim 13, wherein said light and/or heavy chain variable region comprises a mouse or human framework region.

20. The composition of claim 15, wherein said CD160 binding molecule further comprises a light chain constant region and a CH1 heavy chain constant region.

21. The composition of claim 20, wherein said CD160 binding molecule further comprises a CH2 heavy chain constant region.

22. The composition of claim 21, wherein said CD160 binding molecule further comprises a CH3 heavy chain constant region.

23. The composition of claim 22, wherein said light chain constant region is human or humanized murine, and/or wherein said CH1, CH2, and CH3 heavy chain constant regions are human or are humanized murine.

24. The composition of claim 15, wherein said CD160 binding molecule comprises an antibody, wherein the light chain constant region of said antibody is selected from: IgG Kappa and IgG Lambda, and wherein the heavy chain constant region of said antibody is selected from: IgG1, IgG2, IgG3, and IgG4.

25. A method of treating or preventing a disease or infection comprising:
treating a subject with a CD160 binding molecule, or an expression vector encoding said CD160 binding molecule, and
wherein said subject has, or is suspected to develop, a disease or infection selected from: an inflammatory disease, cancer, a chronic infection, and a refractory infection.

26. The method of claim 25, wherein said CD160 binding molecules is as recited in any of claims 1-12.

27. The method of claim 25, wherein said inflammatory disease is a cardiovascular disease or autoimmune disease.

28. The method of claim 27, wherein said cardiovascular disease is selected from: acute myocardial infarction, heart failure, atherosclerosis, progression of atherosclerotic disease, cardiogenic shock.

29. The method of claim 27, wherein said autoimmune disease is selected from the group consisting of: rheumatoid arthritis, inflammatory bowel disease, septic shock, psoriasis, or said autoimmune disease derives from inflammation post treatment with an immune checkpoint inhibitor.

30. The method of claim 27, wherein said CD160 binding molecule is an antibody or antigen binding portion thereof.

31. The method of claim 30, wherein said antibody or antigen binding portion thereof is a human antibody or antigen binding portion thereof.

32. The method of claim 30, wherein said antibody or antigen binding portion thereof is a humanized antibody or antigen binding portion thereof.

33. The method of claim 25, wherein a sample from said subject is assayed to determine levels of NK cell activation and/or inflammation before and/or after said treating.

34. A method of detecting CD160 in a sample comprising:
a) contacting a sample with the CD160 binding molecule of any of claims 1-12,
wherein said sample is from a subject that has, or is suspected to develop, a disease or infection selected from: an inflammatory disease, cancer, a chronic infection, and a refractory infection,
wherein said sample is suspected of containing CD160, and wherein said CD160 binding molecule forms a complex with said CD160 if present in said sample; and
b) detecting the presence or absence of said complex in said sample.

35. The method of claim 34, wherein said CD160 binding molecule comprises a detectable label.

36. The method of claim 34, further comprising contacting said sample with a conjugate molecule capable of binding to said CD160 binding molecule, wherein said conjugate molecule comprises a detectable label.


SEQUENCE LISTING (20 sequences)

SEQ ID NO: 1 - protein, 127 aa, Mus musculus
MVSTSQLLGLLLFWTSASRCDIVMTQSPATLSVTPGDRVSLSCRASQSISDHLHWYQQKSHESPRLLIKYASQSISGIPSRFSGSGSGSDFTLSINTVEPEDVGVYYCQHGHSFPFTFGSGTKLEIK

SEQ ID NO: 2 - protein, 11 aa, Mus musculus
RASQSISDHLH

SEQ ID NO: 3 - protein, 7 aa, Mus musculus
YASQSIS

SEQ ID NO: 4 - protein, 9 aa, Mus musculus
QHGHSFPFT

SEQ ID NO: 5 - protein, 136 aa, Mus musculus
MKVLSLLYLLTAIPGILSDVQLQESGPGLVKPSQSLSLTCSVTGSSITSDYYWSWIRQFPGNKLEWMAYITYDGITNYSPSLKNRISITRDTSKNQFFLKLNSVTTEDTATYYCARDHYYNYFDYWGQGTTLTVSS

SEQ ID NO: 6 - protein, 6 aa, Mus musculus
SDYYWS

SEQ ID NO: 7 - protein, 16 aa, Mus musculus
YITYDGITNYSPSLKN

SEQ ID NO: 8 - protein, 9 aa, Mus musculus
DHYYNYFDY

SEQ ID NO: 9 - protein, 127 aa, Mus musculus
MVFTPQILGLMLFWISASRGDIVLTQSPPTLSVTPRDSVSLSCRASQSISNNLHWYHQKSHESPRLLIKYASQSISGIPSRFSGSGSGTDFTLSINSVETEDFGMYFCHQSNSWPHTFGGGTKLEIK

SEQ ID NO: 10 - protein, 11 aa, Mus musculus
RASQSISNNLH

SEQ ID NO: 11 - protein, 7 aa, Mus musculus
YASQSIS

SEQ ID NO: 12 - protein, 9 aa, Mus musculus
HQSNSWPHT

SEQ ID NO: 13 - protein, 135 aa, Mus musculus
MECNWILPFILSVTSGVYSQIQLQQSGAVLARPGASVKLSCKASGYTFTTYWMQWVKQRPGQGLEWIGAIYPGDGDSRYTQKFKGKATLTADKSSSTAYIQLSNLVSEDSAVYYCARRLGGFDYWGQGTTLTVSS

SEQ ID NO: 14 - protein, 4 aa, Mus musculus
YWMQ

SEQ ID NO: 15 - protein, 17 aa, Mus musculus
AIYPGDGDSRYTQKFKG

SEQ ID NO: 16 - protein, 7 aa, Mus musculus
RLGGFDY

SEQ ID NO: 17 - DNA, 354 bp, Mus musculus
gatgtacagcttcaggagtcaggacctggcctcgtgaaaccttctcagtctctgtctctcacctgctctgtcactggctcctccatcaccagtgattattactggagctggatccggcagtttccaggcaacaaactggaatggatggcctacataacctacgacggtatcactaactacagcccatctctcaaaaatcgaatctccatcactcgtgacacatctaagaaccagtttttcctgaagttgaattctgtgactactgaggacacagctacatattactgtgcaagagatcattactacaactactttgactactggggccaaggcaccactctcacagtctcctca

SEQ ID NO: 18 - DNA, 321 bp, Mus musculus
gacattgtgatgactcagtctccagccaccctgtctgtgactccaggagatagagtctctctttcctgcagggccagccagagtattagcgaccacttacactggtatcaacaaaaatcacatgagtctccaaggcttctcatcaaatatgcttcccaatccatctctgggatcccctccaggttcagtggcagtggatcagggtcagatttcactctcagtatcaacactgtggaacctgaagatgttggagtgtattactgtcaacatggtcacagctttccattcacgttcggctcggggacaaagttagaaataaaa

SEQ ID NO: 19 - DNA, 405 bp, Mus musculus
atggaatgtaactggatacttccttttattctgtcagtaacttcaggtgtctactcacagattcagctccagcagtctggggctgtactggcaagacctggggcttcagtgaagttgtcctgcaaggcttctggctacacctttacgacctactggatgcagtgggtaaaacagaggcctggacagggtctggaatggattggggctatttatcctggagatggtgatagtaggtacactcagaagttcaagggcaaggccacattgactgcagataaatcctccagcacagcctacatccaactcagcaacttggtttctgaggactctgcggtctattactgtgcaagacgactgggggggtttgactactggggccaaggcaccactctcacagtctcctca

SEQ ID NO: 20 - DNA, 381 bp, Mus musculus
atggttttcacacctcagatacttggacttatgcttttttggatttcagcctccagaggtgatattgtgctaactcagtctccacccaccctgtctgtgactccaagagatagcgtcagtctttcctgcagggccagccaaagtattagcaacaacctccactggtatcaccaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcatcagagtaacagctggcctcatacgttcggaggggggaccaagctggaaataaaa